(19) United States
(12) Patent Application Publication
(10) Pub. No.: US 2022/0004658 A1
(43) Pub. Date: Jan. 6, 2022

(54) CARDIAC MONITORING SYSTEM

(71) Applicants: Johanna Paola Contreras, New York, NY (US); Apratim Dutta, New York, NY (US)

(72) Inventors: Johanna Paola Contreras, New York, NY (US); Apratim Dutta, New York, NY (US)

(21) Appl. No.: 17/365,735

(22) Filed: Jul. 1, 2021

Related U.S. Application Data
(60) Provisional application No. 63/046,900, filed on Jul. 1, 2020.

(51) Int. Cl. G06F 21/62; G16H 10/60; G06F 21/32; H04L 9/08; G06K 9/00; A61B 5/318

(52) U.S. Cl. CPC G06F 21/6245; G16H 10/60; G06F 21/32; H04L 9/088; G16H 40/67; G06K 9/00288; G06K 9/00617; A61B 5/318; G06K 9/00087

(57) ABSTRACT

An identification system including a first biometric identifier, a second biometric identifier, a first cardiac identifier logically related to the first biometric identifier, a second cardiac identifier logically related to the second biometric identifier, where the identity of a user is verified using the biometric identifiers and the cardiac identifiers.

### CROSS-REFERENCE TO RELATED APPLICATIONS

The present disclosure is a continuation of U.S. Application No. 63/046,900, filed on Jul. 1, 2020, which is incorporated by reference in its entirety.

### BACKGROUND OF THE INVENTION

Millions of confirmed cases of COVID-19 have been identified, including hundreds of thousands of deaths having been reported to the World Health Organization (“WHO”). The risk of severe disease and death has been highest in elderly people and in persons with underlying noncommunicable diseases (NCDs), such as hypertension, cardiac disease, chronic lung disease and cancer. Limited data describe clinical manifestations of COVID-19 that are generally milder in children compared with adults, but also show that some children do require hospitalization andintensive care.

However, many patients develop a high or hyperinflammatory state secondary to a cytokine storm that is macrophage activation syndrome, or cytokine release syndrome and as aconsequence they develop a severe vasodilatory shock that is difficult to manage and requiresseveral strategies including immune-suppressive therapy, inotropic support, metabolic support andin severe cases cardiac circulatory support including extracorporeal membrane oxygenation. [0003] The management of this becomes very challenging without objective monitoring.Therefore, a need exists for a temporary monitoring solution.

### SUMMARY OF THE INVENTION

Systems, methods, features, and advantages of the present invention will be or will become apparent to one with skill in the art upon examination of the following figures and detailed description. It is intended that all such additional systems, methods, features, and advantages be included within this description, be within the scope of the invention, and be protected by the accompanying claims.

One embodiment of the current disclosure includes an identification system including, a first biometric identifier, a second biometric identifier, a cardiac identifier logically related to the first and second biometric identifiers, where the identity of a user is verified using the biometric identifiers and the cardiac identifier.

### BRIEF DESCRIPTION OF THE DRAWINGS

The accompanying drawings, which are incorporated in and constitute a part of thisspecification, illustrate an implementation of the present invention and, together with the description, serve to explain the advantages and principles of the invention. In the drawings:

FIG. 1 depicts one embodiment of a row identification system consistent with the present invention;

FIG. 2 depicts one embodiment of a cardiac monitoring unit;

FIG. 3 depicts one embodiment of a communication device consistent with the present invention;

FIG. 4 depicts a sensor system implanted in a right ventricle of a heart continuouslymonitors physiological parameters of a patient;

FIG. 5 depicts a sensor system is combined with a pacemaker and implanted in a heart;

FIG. 6 depicts one embodiment of a wireless communication module connected to the leads;

FIG. 7 depicts a communication diagram for the cardiac monitoring system;

FIG. 8 depicts a schematic representation of an analysis performed by the informationanalysis unit on the information stored in the centralized database;

FIG. 9 depicts a schematic representation of an encryption process performed by thereporting unit;

FIG. 10 depicts a schematic representation of a process to generate a cardiac identifier fora user; and

FIG. 11 depicts schematic representation of a process to verify a user of a device usingcardiac information.

### DETAILED DESCRIPTION OF THE INVENTION

Referring now to the drawings which depict different embodiments consistent with the present invention, wherever possible, the same reference numbers will be used throughout the drawings and the following description to refer to the same or like parts.

The cardiac monitoring unit will monitor oxygen level, cardiac output and pressure in patients in the intensive care unit admitted with severe COVID-19 infection, avoiding need for placement of Swan-Ganz catheter, minimizing blood drawn and having an objective estimated ofpatient oxygenation that is reliable and easily transmitted to a wireless device. The traditional practice of placement of a Swan-Ganz Catheter requires close contact with patient, repetitive calibration and requires nursing staff to take blood samples from catheter every time the clinician requires a measurement of mixed venous Oxygen. The traditional approach also requires the clinician to actively manipulate the catheter to obtain measurement of pressure and cardiac output. Patients receiving a Swan-Ganz catheter are at a high risk of causing systemic infection, consequently, the catheter should not remain inserted into the patient longer than 7 days. If after 7 days, the patient still requires monitoring, then the patient will have to have a new catheter inserted. On the other hand, after a few days of having the catheter in place the information may not be as accurate and it can become difficult to obtain wedge pressure.

Patients with moderate to severe COVID-19 infection and lung disease, can have varying manifestations of the disease, from mind pneumonitis to full ARDS (acute respiratory distress syndrome), and some patients can have normal tissue oxygenation while showing low oxygen saturation by traditional external oxygen saturation monitoring which are placed on patients finger or ear lop, these traditional oxygen monitors can capture inaccurate true oxygen saturation. It has been described that it may be two different phenotypes of lung disease, Type L (of Type 1) and Type H (or Type 2). Patients typically present with Type L disease, which is characterized by normal lung compliance and gas volume in the present of hypoxemia. These patients may improve, or they may worsen. About 20-30% of patients had, or evolved to Type H disease, characterized by decreased lung compliance and increased edema and lung weight. The transition from Type L to Type H may be due to the evolution of the COVID-19 pneumonia on one hand and the injury attributable to high-stress ventilation on the other. Type L patients can still be placed on the ventilator bybut with higher tidal volumes with lower positive end expiratory pressure (PEEP), while Type H patients should be treated as severe ARDS, including higher PEEP, if compatible with hemodynamics, prone positioning and ECMO.

As one having ordinary skill in the art will recognize, having an objective measurement of central mixed venous saturation will help differentiate between Type L and Type H and provide the best method of treatment and will allow for a determination of what patients need escalation of care to ECMO or a different strategy of therapy. The cardiac monitor will allow early identification of patient transitioning from Type L to Type H and intervene early, thus prevention complication and death. Also, the monitor may allow physicians to allow a more permissive hypoxia and prevent intubation since some of these cases intubation and mechanical ventilation can worsen clinical status. Using the cardiac monitor as a temporary monitor is convenient and could also be used in non-intensive care unit setting since it does not require specialized monitoring or specialized nursing skills. Also, the data is remote, so it can be used in regular COVID-floor units, facilitating work and oxygen monitoring remotely. Temporary monitoring with the cardiac unit will help monitor patients with moderate COVID-19 infection and that can be treated at home but currently, because of the lack of reliable and available monitors, are being treated at the hospital. They can have the monitor at home during infection to make sure that they maintain adequate oxygenation and identify early deterioration so patients can come to the hospital for further treatment if needed. The temporary monitor will be also an essential tool to monitor response to therapy e.g. Infusion of convalescent plasma or antiretrovirals.

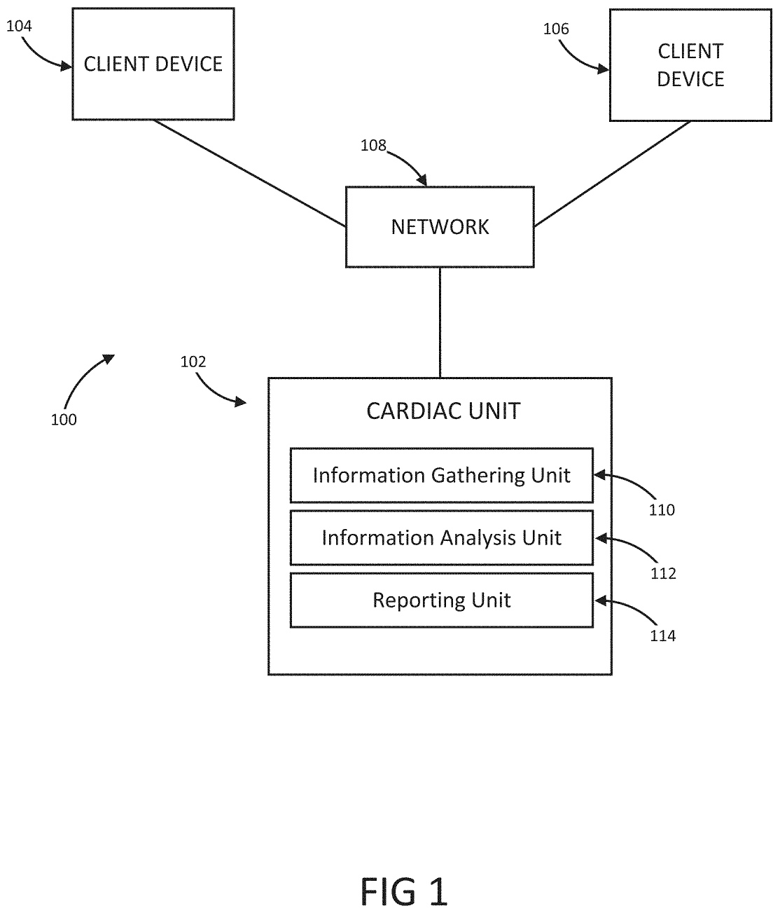

FIG. 1 depicts one embodiment of a row identification system 100 consistent with the present invention. The row identification system 100 includes a row identification device 102, a communication device 1 104, a communication device 2 106 each communicatively connected via a network 108. The cardiac monitoring system 100 further includes an information gathering unit 110, an information analysis unit 112, and a reporting unit 114.

The information gathering unit 110 and information analysis unit 112 may be embodied by one or more servers. In one embodiment, the network 108 is a cellular network, a TCP/IP network, or any other suitable network topology. In another embodiment, the row identification device may be servers, workstations, network appliances or any other suitable data storage devices. In another embodiment, the communication devices 104 and 106 may be any combination of cellular phones, smart phones, telephones, tablet, personal data assistants, or any other suitable communication devices. In one embodiment, the network 108 may be any private or public communication network known to one skilled in the art such as a local area network (“LAN”), wide area network (“WAN”), peer-to-peer network, cellular network or any suitable network, using standard communication protocols. The network 108 may include hardwired as well as wireless branches.

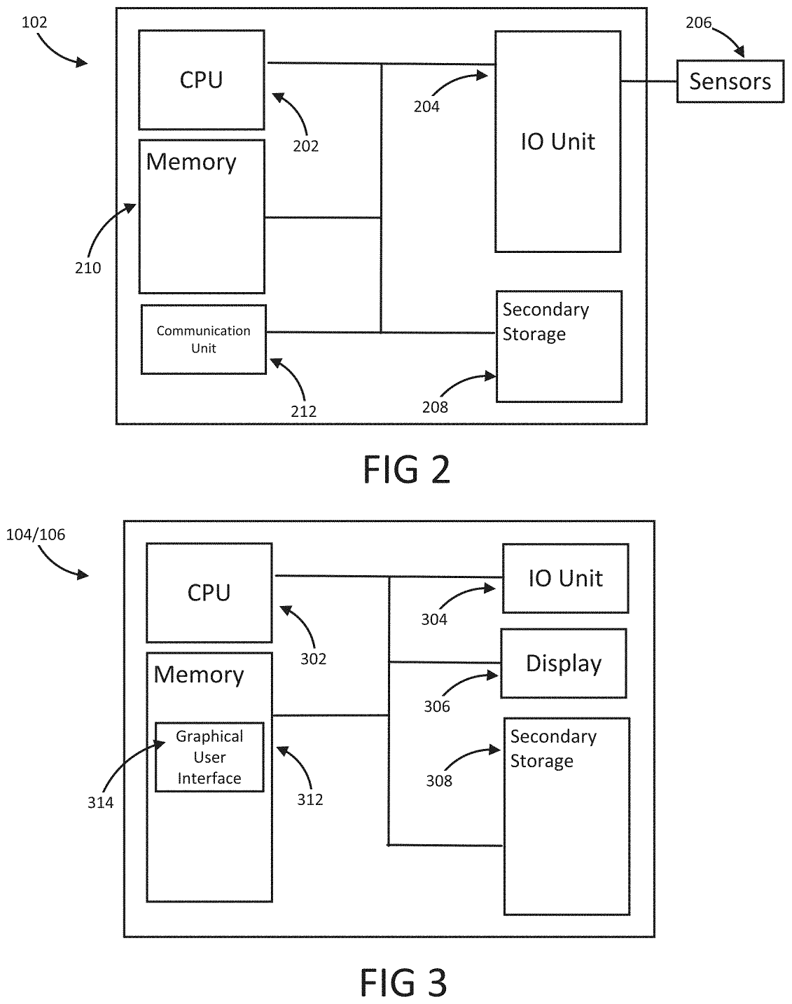

FIG. 2 depicts one embodiment of a cardiac monitoring unit 102. The cardiac monitoring unit 102 includes an I/O device 204 connected to at least one sensor 206, a processor 202, and a secondary storage 208 and a memory 210 In one embodiment, the processor 202 may be a central processing unit (“CPU”), an application specific integrated circuit (“ASIC”), a microprocessor or any other suitable processing device. The memory 210 may include a hard disk, random access memory, cache, removable media drive, mass storage or configuration suitable as storage for data, instructions, and information. In one embodiment, the memory 208 and processor 202 may be integrated. The memory may use any type of volatile or non-volatile storage techniques and mediums. A communication unit 212 allows for communication with external devices via wireless protocols including near field communication, Bluetooth communication or any other wireless communication.

FIG. 3 depicts one embodiment of a communication device 104/106 consistent with the present invention. The communication device 104/106 includes a processor 302, a network I/O Unit 304, an image display unit 306, a secondary storage unit 308 including an image storage device 310, and memory 312 running a graphical user interface 314. In one embodiment, the processor 302 may be a central processing unit (“CPU”), an application specific integrated circuit (“ASIC”), a microprocessor or any other suitable processing device. The memory 312 may include a hard disk, random access memory, cache, removable media drive, mass storage or configuration suitable as storage for data, instructions, and information. In one embodiment, the memory 312 and processor 302 may be integrated. The memory may use any type of volatile or non-volatile storage techniques and mediums. The network I/O device 304 may be a network interface card, a plain old telephone service (“POTS”) interface card, an ASCII interface card, or any other suitable network interface device.

In one embodiment, the network 108 may be any private or public communication network known to one skilled in the art such as a Local Area Network (“LAN”), Wide Area Network (“WAN”), Peer-to-Peer Network, Cellular network or any suitable network, using standard communication protocols. The network 108 may include hardwired as well as wirelessbranches.

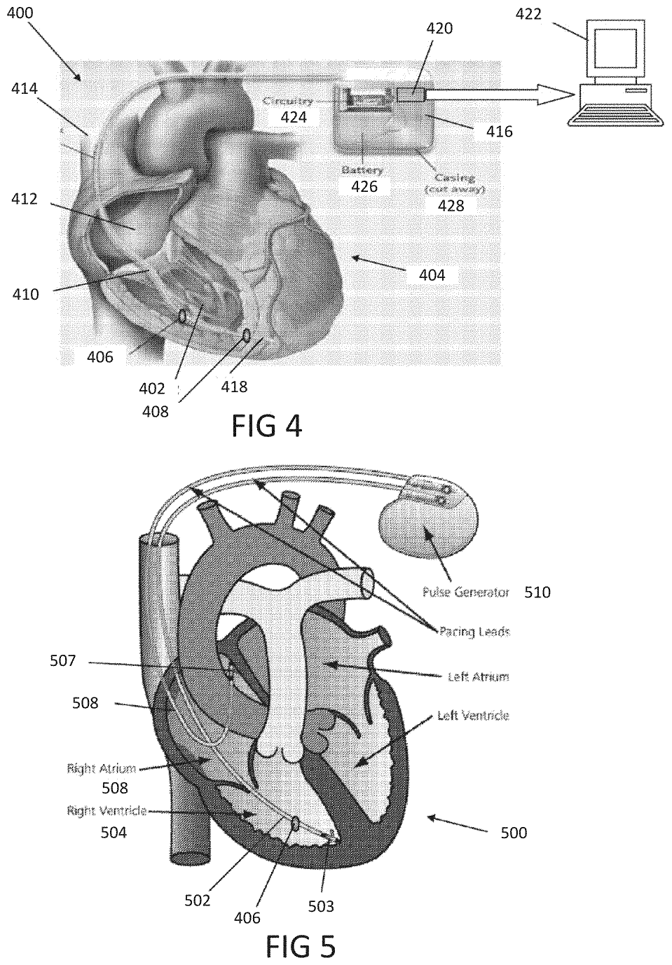

Referring to FIG. 4, a sensor system 400 implanted in a right ventricle 402 of a heart 404 continuously monitors physiological parameters of a patient. An oxygen sensor 406 measures the central mixed venous oxygen level (MvO2) or the central mixed venous oxygen saturation (CvO2) and the percent oxygen saturation in right ventricular blood. Simultaneously, a pressure sensor 408 measures the central venous fluid pressure and pulse pressure in the right ventricle and the maximum positive and negative rate of change of the pressure during the cardiac cycle (dP/dt). In some embodiments, pressure sensor 408 also measures an intracardiac electrocardiogram and an impedance of heart and lung tissue. In other embodiments, onlyoxygen sensor 406 is used. The use of an oxygen sensor alone is useful, for instance, formonitoring patients with pulmonary hypertension or with primary oxygenation impairment.

These physiological parameters provide data that can be used to identify and monitororgan perfusion, congestion in the chest cavity, and the degree of compensation or decompensation in patients with chronic cardiopulmonary failure or other types of cardiopulmonary disease. When coupled with cardiac output measurements, this data enables thecalculation of oxygen transport and oxygen consumption; early identification of impending oractual global tissue hypoxia; a determination of the cause of a hypoxic episode; an assessment ofthe response of a patient to a treatment of hypoxia; and a prediction of patient survival based onan underlying cause of a hypoxia episode and on the patient's response to the hypoxia treatment.

Oxygen sensor 406 is approximately less than 1 cm in diameter and is positioned alongthe length of a lead 410 that passes through a right atrium 412 and a superior vena cava 414 atabout 3-4 cm above the tip of the lead. Pressure sensor 408 is positioned toward the end of lead 110, embedded in the wall of the right ventricle towards the apical septum 402. Lead 410 and sensor 406 are inserted intravenously into the right ventricle through the subclavian or cephalic vein of the patient. Lead 410 connects to a control module 416 positioned in a subcutaneous device pocket in the sub clavicular region of the patient, which pocket is formed by a small cutaneous incision, as in currently performed during the implantation of a pacemaker. Lead 410 is between 5-7 mm thick and is typically about 5 mm thick. A tip of 418 of lead 410 is anchored in the myocardium of heart 404 by soft tines or a tiny screw (not shown). A steroid elutes fromtip 418 to decrease inflammation at the tip-myocardium interface, thus improving the chronicity of sensor system 400. As a result, the sensor system 400 is able to remain implanted for long periods of time, allowing longer monitoring of physiological parameters.

Measurement data is transmitted from oxygen sensor 406 and pressure sensor 408 to control module 416 along lead 410. Control module 416 includes a wireless communication module 420, such as an antenna coil. Communication module 420 wirelessly communicates the measurement data to a remote computer 422 for display, storage, or processing. Computer 422 may be, for instance, a clinician's computer, a patient's computer, or a handheld computing device. Communication between control module 416 and computer 422 may be periodic or upon request by computer 422. For instance, computer 422 may calculate both a continuous CvO2 level and an average CvO2 level at a preselected timing interval. Also, once a baseline CvO2 of the patient is obtained, an alarm setting can be programmed that will be activated at predetermined levels of CVO2, thus allowing early recognition of a decline or a decompensated status.

Control module 416 also includes control circuitry 424 that controls the operation of sensor 406 and communication module 420. A lithium battery 426 in control module 416 supplies power to control circuitry 424, communication module 420, and sensor 406. The lifetime of battery 426 is typically in the range of 5-10 years and depends on factors such as the output voltage of control module 416, the resistance of lead 410 and sensor 416, and thefrequency and duration of use of the battery 426. The components in control module 108 are enclosed in a biocompatible casing 428.

Oxygen sensor 406 and pressure sensor 408 are hermetically sealed devices made of titanium, iridium, or another biocompatible material that is pharmacologically inert, nontoxic, sterilizable, and able to function in the environmental conditions of the body. Ideally the material is not affected by stress cracking or metal ion oxidation. Circuitry in sensor 406 and circuitry in sensor 408 control the operation of measurement devices housed in sensors 406 and 408 and control the communication between the sensors and control module 416.

A light emission module in oxygen sensor 406 includes a red (660 nm) and/or infrared (880 nm) light emitting diode (LED) hermetically sealed in a sapphire capsule. The LED emits light which illuminates blood in the right ventricle. The amount of light reflected by the blood, which is indicative of the oxygen saturation (i.e., the CvO2) is detected by a photodetector. A titanium pressure sensing membrane mounted on pressure sensor 408 measures fluid pressureand pulse pressure in the right ventricle or right atrium.

A set of electrodes mounted on the external surface of pressure sensor 408 measures the impedance of tissue in the chest cavity, such as cardiac tissue and pulmonary tissue, at a digital rate of 128 Hz. Impedance measurements allow for portioned analysis of contractile cardiac function and pulmonary ventilation function. Average pulmonary impedance, e.g., averaged over a period of 72 hours or more, provides a baseline value against which an instantaneous impedance measurement can be compared. Signal processing of the impedance data allows deviations from baseline impedance values to be detected. For instance, a decrease in lung impedance is indicative of increasing fluid content and congestion in the lungs, which can lead tocongestive heart failure.

In some embodiments, the sensor system is integrated with another implantable diagnostic or therapeutic device, such as a prophylactic implantable cardioverter defibrillator (ICD), a biventricular ICD, or a permanent pacemaker (PPM). In general, when the sensor system is integrated with another implantable device, certain structures (e.g., lead 410 in FIG. 1) may be shared between either or both of sensor 406 or sensor 408 and the other implantable device.

Referring to FIG. 5, a sensor system is combined with a pacemaker and implanted in a heart 500. Oxygen sensor 406 is positioned along a ventricular lead 502 of the pacemaker; pressure sensor 408 is positioned at the end of the lead 410. Ventricular lead 502 is anchored in the myocardium of a right ventricle 504 by an anchor 503. An atrial lead 506 of the pacemaker isanchored in the myocardium of a right atrium towards the right interatrial septum 508 by an anchor 507. In some instances, a sensor system such as that shown in FIG. 1 is later upgraded to include a pacemaker (i.e., to become sensor system 300) if a patient's illness evolves to indicate the use of a pacemaker. In other instances, an existing pacemaker is upgraded to include sensors 406 and 408.

In some embodiments, pressure sensor 408 is positioned at the end of atrial lead 506. In some instances, atrial lead 506 is directed toward the base of the inter-atrial septum (not shown) such that pressure sensor 408 is embedded in the wall of the right atrium. The measurements of the right atrial pressure provided by the pressure sensor 408 located on the right atrial lead generally are more accurate than measurements of the right ventricular pressure provided by a pressure sensor located on a right ventricular lead (e.g., sensor 408 in FIG. 1). The placement of both an atrial lead and a ventricular lead is a more invasive procedure (such as a transseptal puncture) than the placement of only a ventricular lead. However, when the sensor system isused in conjunction with a pacemaker (e.g., a dual chamber pacemaker, a PPM/ICD, or a BivICD, an atrial lead and a ventricular lead are already used and thus no additional interventionoccurs.

Referring to FIGS. 4 and 5, a pulse generator 510 is implanted in a subcutaneous devicepocket in the subcutaneous region of a patient and connects to ventricular lead 502 and atrial lead 506. Pulse generator 510 includes a sensor module, a pacemaker module, and a lithium battery. Sensor module 512 controls the operation of sensors 406 and 408 and receives measurement datafrom sensors 406 and 408 via lead 502. Sensor module includes a wireless communication module 420 that communicates the measurement data to a remote computer. Pacemaker module sends electrical pacing signals along ventricular lead 502 and atrial lead 506. Pacemaker module controls the pacing of the pacemaker according to a predetermined algorithm that takes into account physiological parameters including heart rate, QRS duration and morphology, PR intervals, and CvO2 levels. Battery provides power to sensor module, pacemaker module, communication module, and sensors 406 and 408.

In one embodiment, the anchor 503 that anchors the lead 502 into the myocardium of a right ventricle 504 is detachable form the lead 502 for removal from the heart. In another embodiment, the tip of the lead 502 connected to the myocardium is broken off to allow the lead 502 to be removed from the heart. In another embodiment, the anchor 503 the anchor is a separate unit from the lead 502 with the anchor 503 separating from the lead 502 when a specificmotion, i.e. twisting, bending, etc. is performed on the lead 502. In another embodiment, the anchor 503 may be retracted from the myocardium such that the anchor 503 and lead 502 may beremoved together.

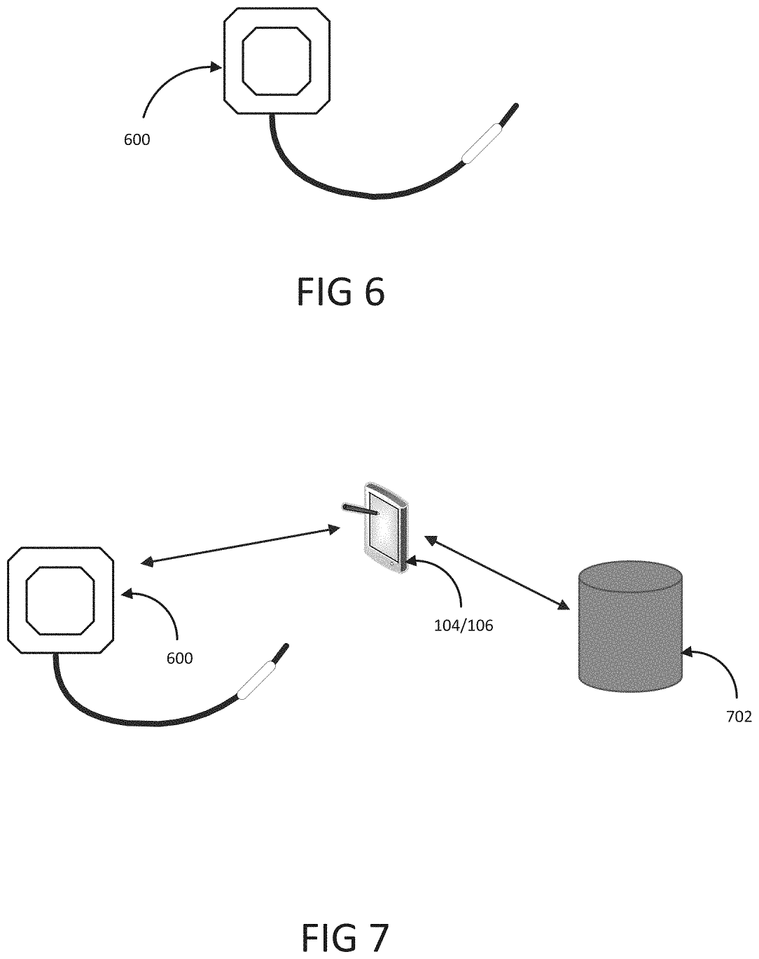

FIG. 6 depicts one embodiment of a wireless communication module 600 connected tothe leads 410. The module 600 includes the processor 202 and memory 210 with the I/O unit 204 controlling power and communication to the leads 410. A multi-dimensional array or database of information is stored in the memory 210. The array may include biometric information gathered from the leads 506 and stored in the multi-dimensional array or database. The communication unit 212 may be a near field communication unit that communicates using Bluetooth and/or near field communications. In one embodiment, a client device, including a smart phone or tablet is place in the vicinity of the patient. The smart phone or tablet initiate communication with the device to gather information from the device including information from the multi-dimensional array and operational information related to the device. In another embodiment, the module activates a passive card placed within the vicinity of the module 600. The module 600 may transmit information stored in the multi-dimensional array or to the card for storage. In another embodiment, the battery in the module 600 is wireless charged using magnetic resonance or inductance.

FIG. 7 depicts a communication diagram 700 for the cardiac monitoring system. The module 600 communicates with the client device 104/106. The information received into the client device 104/106 is encrypted and sent to a central database 702. The centralized database stores biometric information on a plurality of patients with different conditions. A first portion of the database 702 is used to store individual patient information. A second portion of the information is used to store a cleaned version of patients information. The cleaned version of the database may remove all identifying information related to the patient. In one embodiment, the central database gathers information from physicians on an individual patient's condition, outcome, side effects and other medical observations The central database 702 may alsotransmit and receive addition patient information from other locations using the encryption methods discussed herein. In one embodiment, application programmable interfaces (“API”) are used to gather information from third party data locations including, but not limited to, health care providers insurance companies.

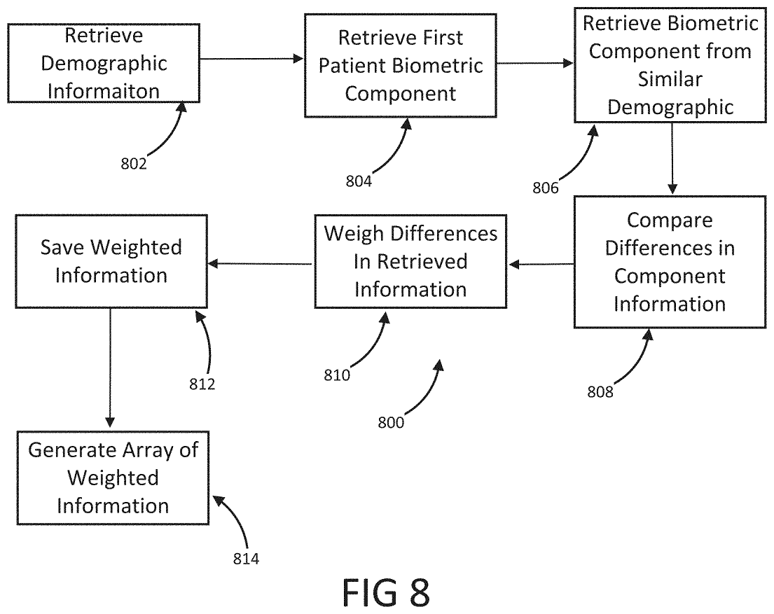

FIG. 8 depicts a schematic representation of an analysis performed by the information analysis unit 112 on the information stored in the centralized database. In step 802, demographic information on a patient is retrieved from the central database 702. Demographic information may include age, race, gender, pre-existing conditions, or any other information on the demographics of a patient. In step 804, a first patient biometric information is gathered from the central database 702. The first patient biometric information may be any biometric information related to the heath, race or age-related information of the patient. In step 806, the information analysis unit 112 gathers information related to patients with similar demographic information. In step 808, the retrieved demographic information is comparted to the patient's information. In step 810, the patient's information is weighted against the retrieved demographics. In step 812, the weighted information is saved and the process resumes gathering demographic information. In step 814, a weighted array of biometric information representing a comparison of the patient's biometric information compared to others in the same demographic group.

In one embodiment, the information analysis unit 112 uses right ventricular pressures, right atrial pressure, and mixed venous oxygen saturation, to generate a better understanding of patient hemodynamic status at any time and able to calculate cardiac output, and tissue oxygenations. The information analysis unit 112 may combine this information with currently known information including, but not limited to, percentage to RV pacing, the burden of Atrial fibrillation or ectopy, activity level, thoracic impedance, respiratory patterns and ventricular arrhythmias. These measurements will provide a clinician a more accurate assessment of patient hemodynamics status to make the best determination of therapies. This hemodynamic data will provide feedback on patients with electrical disturbance to determine the severity of arrhythmia and the need for ICD shock, thus either ensuring appropriate ICD therapy or prevention inappropriate ICD therapies in patients, a therapy that if applied without indication could be harmful to patients.

Having a combination of hemodynamics e.g. right ventricular pressures, right atrial pressure and mixed venous oxygen saturation, provides a better understanding of patient hemodynamic status at any time, and also allows for the calculation of cardiac output and tissue oxygenations. These measurements combined with current available measurements in ICDs and BiVICD, which includes, but is not limited to, percentage to RV pacing, burden of atrial fibrillation or ectopy, activity level, thoracic impedance, respiratory patterns and ventricular arrhythmias; will provide a more accurate assessment of patient hemodynamics status to make the best determination of therapies. In one embodiment, an index that is combined with current measurement plus the oxygen monitor measurement that generates an alert for imminent acute decompensation, hemodynamic imbalance, predictor of further decompensation and clinical stability of patient. This hemodynamic data will provide a feedback on patient with electrical disturbance to determine severity of arrhythmia and need for ICD shock, thus preventing inappropriate ICD therapies in patients, therapy that if apply without indication could be harmful to patients.

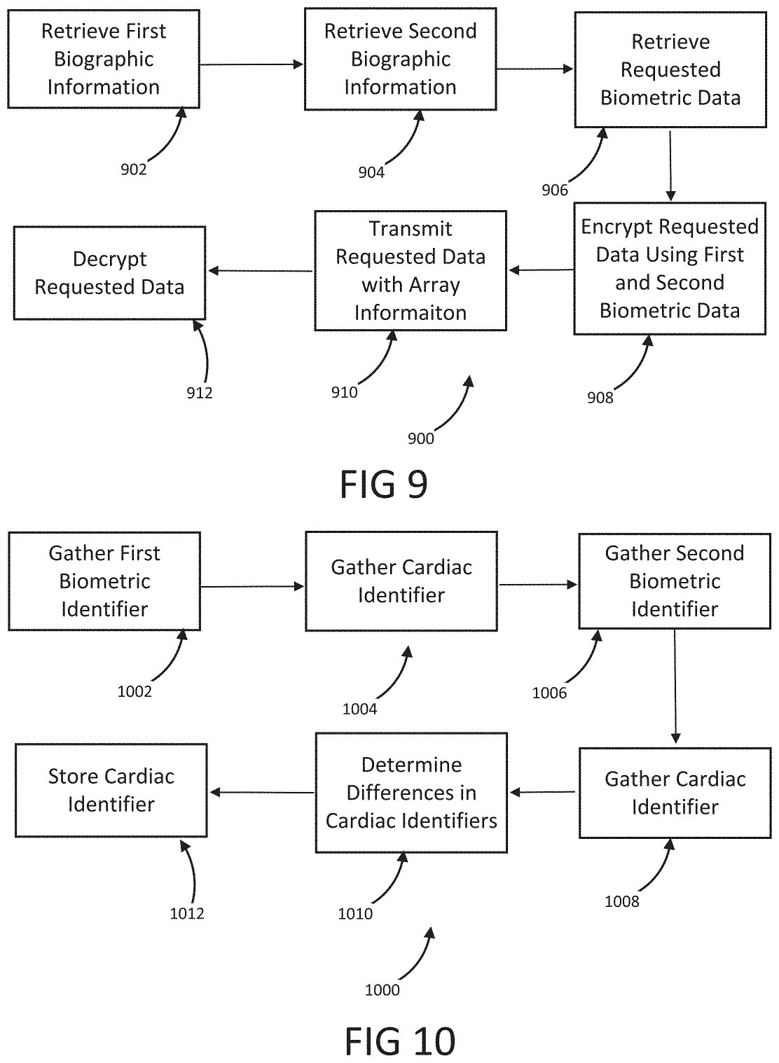

FIG. 9 depicts a schematic representation of an encryption process performed by the reporting unit 114. In step 902, the reporting unit 114 retrieves a first piece of biometric information from the database at a specific date and time represented by a key indicator in the database. The first piece of biometric information is a number representing a reading from one of the sensors in the leads 410. In step 904, the reporting unit selects a second piece of biometricinformation from the database at a second date and time represented by a second key indicator in the database. In step 906, the reporting unit 114 retrieves the requested biometric information to be send out of the unit along with the first key indicator and second key indicator. In step 908, the requested information is encrypted using the first biometric data and second biometric data as encryption keys. In step 910, the encrypted information and key indicators are sent to the receiving device. In step 912, the receiving device uses the key indicators to retrieve the key to decrypt the information. In another embodiment, the encryption information is stored on a smartcard that is manipulated by the monitoring unit based on recorded biometric information. The card may be presented to a device to unencrypt data received from the monitoring unit.

FIG. 10 depicts a schematic representation of a process to generate a cardiac identifier for a user. In step 1002, a first biometric identifier is received. The first biometric identifier may be any biometric identifier that is unique to the user including, but not limited to, a fingerprint, facial recognition, iris recognition or any other identifying biometric feature. In step 1004, a cardiac identifier is received using a heat monitoring device such as an electrocardiogram (EKG). As an illustrative example, a user may place their hands on a device attached to a cellular phone that generates an EKG signal that is stored in the memory of the phone. The phone may also retrieve the biometric identifiers. In step 1006, a second biometric identifier, different than the first biometric identifier is retrieved. In step 1008, the cardiac identifier is retrieved from the EKG device a second time and compared to the first cardiac identifier to confirm the two identifiers are the same. In step 1010, differences in the cardiac identifiers are identified and the cardiac identifier is normalized. In step 1012, the cardiac identifiers are stored with the biometric indicators.

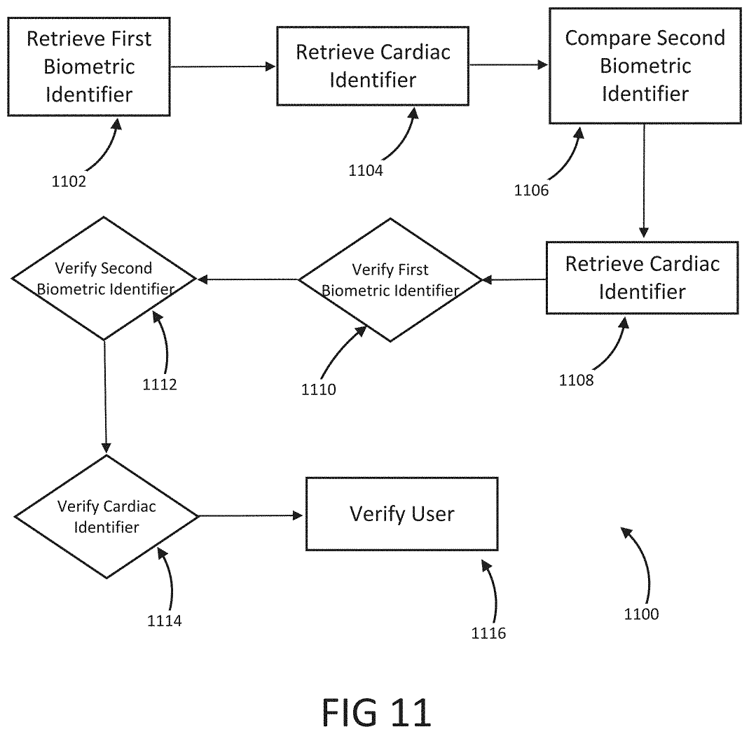

FIG. 11 depicts schematic representation of a process to verify a user of a device using cardiac information. In step 1102, a first biometric identifier is retrieved from the user. In step 1102, the first biometric identifier is retrieved from the user. In step 1104, the cardiac identifieris retrieved from the user. In one embodiment, the first biometric indicator is retrieved simultaneously with the cardiac information. In step 1106, the second biometric identifier is retrieved from the user. In step 1108, the cardiac identifier is retrieved a second time. In one embodiment, the cardiac identifier is retrieved simultaneously with the second biometric identifier. In step 1110, the first biometric identifier is compared to the first biometric identifierstored in the database. In step 1112, the second biometric identifier is compared with the second biometric identifier stored in the identifier. In step 1114, the cardiac identifier is compared to thetwo cardiac identifiers retrieved from the user. In step 1116, the user is verified if the cardiac identifier and two biometric identifiers match the information stored in the database for the user.

In one embodiment, once a user is authenticated, identification information may be incorporated into a digital record including, but not limited to, a blockchain. In another embodiment, the blockchain verification tokens are compliant with W3 standards or other heath care validation standards. In another embodiment, the user's medical records and vital statistics are stored in the user's blockchain where they can be validated for third parties requiring the medical information. In one embodiment, the verified information may be used to produce a bar code or a QR that uses gathered biometric information as the encryption key.

While various embodiments of the present invention have been described, it will be apparent to those of skill in the art that many more embodiments and implementations arepossible that are within the scope of this invention. Accordingly, the present invention is not tobe restricted except in light of the attached claims.

What is claimed:

1. An identification system including:
a first biometric identifier;
a second biometric identifier;
a first cardiac identifier logically related to the first biometric identifier;
a second cardiac identifier logically related to the second biometric identifier,
wherein,
the identity of a user is verified using the biometric identifiers and the cardiac identifiers.

2. The system of claim 1, wherein the first biometric identifier is gathered simultaneously with the first cardiac identifier.

3. The system of claim 1, wherein the second biometric identifier is gathered simultaneously with the second cardiac identifier.

4. The system of claim 1, wherein the first biometric identifier is one of a fingerprint, a facial feature or a iris pattern.

5. The system of claim 1, wherein the second is one of a fingerprint, a facial feature or a iris pattern.

6. The system of claim 1, wherein the first cardiac identifier is at least one point on an electrocardiogram.

7. The system of claim 6, wherein the second cardiac identifier is the least one point on an electrocardiogram.

8. The system of claim 1, wherein the first and second cardiac identifiers are normalized.

9. The system of claim 8, wherein the normalized cardiac identifiers are logically related to the first biometric identifier and the second biometric identifier.

10. The system of claim 1, wherein the biometric and cardiac identifiers are gathered from a mobile communication device.

11. A method of identifying a user of a device, the method including the steps of:
gathering a first biometric identifier;
gathering a second biometric identifier;
logically relating a first cardiac identifier to the first biometric identifier;
logically relating a second cardiac identifier to the second biometric identifier,
verifying the user using the biometric identifiers and the cardiac identifiers.

12. The method of claim 11, wherein the first biometric identifier is gathered simultaneously with the first cardiac identifier.

13. The method of claim 11, wherein the second biometric identifier is gathered simultaneously with the second cardiac identifier.

14. The method of claim 11, wherein the first biometric identifier is one of a fingerprint, a facial feature or a iris pattern.

15. The method of claim 11, wherein the second is one of a fingerprint, a facial feature or a iris pattern.

16. The method of claim 11, wherein the first cardiac identifier is at least one point on an electrocardiogram.

17. The method of claim 16, wherein the second cardiac identifier is the least one point on an electrocardiogram.

18. The method of claim 11, wherein the first and second cardiac identifiers are
